(19) United States
(12) Patent Application Publication
(10) Pub. No.: US 2014/0294941 A1
(43) Pub. Date: Oct. 2, 2014

(54) ANTI-ANGIOGENIC COMPOSITIONS AND THERAPEUTIC APPLICATIONS THEREOF

(71) Applicant: Medestea Research & Production S.P.A., Colleretto Giacosa (IT)

(72) Inventors: Arnaldo CARUSO, Brescia (IT); Dario Di Luca, Masi San Giacomo (IT)

(21) Appl. No.: 14/309,483

(22) Filed: Jun. 19, 2014

Related U.S. Application Data
(62) Division of application No. 12/997,493, filed on Feb. 18, 2011, now abandoned.
(60) Provisional application No. 61/060,962, filed on Jun. 12, 2008.

(51) Int. Cl. C07K 14/005

(52) U.S. Cl. CPC C07K 14/005
USPC 424/450; 514/44 R; 514/13.3

(57) ABSTRACT

There is described the use of the Human herpesvirus type 6 (HHV-6) U94 gene and its product, the protein Rep, expression vectors and pharmaceutical compositions suitable for the delivery of the U94 gene or the protein encoded therefrom to the therapeutic sites, to inhibit angiogenic and lymphangiogenic processes in a subject in need thereof.

The present invention relates to the use of the Human herpesvirus type 6 (HHV-6) U94 gene and its product, the protein Rep, in human therapy. According to the invention, the protein Rep or the Rep-coding human herpesvirus type 6 (HHV-6) U94 gene are delivered to human blood (BEC) and lymphatic (LEC) endothelial cells, whereby angiogenic and lymphangiogenic processes are inhibited. The invention further provides expression vectors and pharmaceutical compositions suitable for the delivery of the U94 gene or the protein encoded therefrom to the therapeutic sites.

### BACKGROUND OF THE INVENTION

Angiogenesis describes the formation of new blood vessels from the preexisting microvasculature. Under physiological conditions, angiogenesis is a highly regulated phenomenon mediated by a tightly controlled and balanced synthesis of numerous proangiogenic and antiangiogenic factors (Hanahan and Folkman, 1996, Cell 86:353-364). In physiological conditions, angiogenesis takes place during embryogenesis and early after birth, as well as in the adult in the context of wound healing and the female reproductive cycle. In physiological conditions, cells are located within 100-200 μm from blood vessels, their source of oxygen. When a multicellular organism is growing, cells induce angiogenesis and vasculogenesis in order to recruit new blood supply. Unregulated angiogenesis is seen in pathological conditions, such as the chronic inflammatory disease psoriasis, infantile haemangiomas, peptic ulcers, ocular neovascularisation, atherosclerosis and career (Mauriz and Gonzalez-Gallego, 2008, J Pharm Sci, Epub ahead of print).

Cancer starts off as small groups of abnormal cells that proliferate rapidly and enlarge. This process is angiogenesis dependent, as a tumor cells located 100 μm away from blood vessels become hypoxic. A tumor cannot grow beyond 1-2 mm in size without an adequate blood supply. Until the tumor cell acquires the ability to produce its own, or to modify its environment to produce angiogenic stimulators de novo, its cells may remain dormant or even disappear owing to other host factors. The progression of a tumor from a dormant to an active status depends on a series of events, including a switch to an angiogenic phenotype (John et al., 2007, Br J Surg 95:281-293). This can be triggered by various signals, including hypoxia, metabolic stress, mechanical stress and immune/inflammatory response. This switch can also be thrown by loss of antiangiogenic factors. To generate capillary sprouts, endothelial cells proliferate, migrate, degrade the basement membrane, and form a structure, that is a new lumen organisation. To stimulate angiogenesis, both tumor cells and host cells secrete a variety of factors. So far, more than a dozen of proangiogenic molecules have been reported, the most potent of which are basic fibroblast growth factor (bFGF) and vascular endothelial growth factor (VEGF).

Antiangiogenic Therapy Inhibits the Sprouting of Blood Bessels and Acts to Arrest the Angiogenic Switch.

The idea of stopping tumor growth and metastases by a therapy that is aimed at interfering with tumor angiogenesis was first published by Folkman (Folkman 1971, N Engl J Med 285:1182-1186). The theoretical advantages of antiangiogenic therapy are based on the observation that endothelial cells are homogeneous, diploid, genetically stable targets, avoiding the selection of drug resistant subpopulations. This is in contrast with the high genetic instability, heterogeneity and mutation rate of tumor cells. Moreover, due to the fact that a single vascular net may support the growth of different populations of tumor cells, the inhibition of the vascular growth may affect the survival of many tumor cells. Today, different angiogenesis inhibitors have been described: interference with the angiogenic factor or their receptors, inhibition of endothelial cell proliferation, inhibition of metalloproteinases (MMPs), inhibition of endothelial cell adhesion and others summarized in Mauriz and Gonzalez-Gallego (J Pharm Sci, 2008). Inhibiting tumor angiogenesis by targeting VEGF signalling is a rational and potentially valuable therapeutic strategy, and today it is one of the most promising new strategies of inhibiting tumor growth and formation of metastases. First drugs have been approved in 2004 and 2006 by FDA. Bevacizumab (Avastin, Genentech), an anti-VEGF-As summarized in Drevs and Schneider (J Int Med 2006, 260:517-529) a humanized recombinant monoclonal antibody has demonstrated, in combination with certain chemotherapy regimens, clinically relevant improvements in survival in colorectal, lung and breast cancer. Anti-tumor activity has been shown with small molecules such as Sorafenib (Nexavar, Bayer) and Sunitinib (Sutent, Pfizer)—both possessing activity against against VEGF receptor tyrosine kinase—when added to the standard treatments of colorectal cancer, renal cell carcinoma, melanoma and gastro-intestinal stroma tumor (GIST).

Problems with Antiangiogenic Therapy z

The current antiangiogenic therapies use inhibitors capable of specifically blocking the activity of a single proangiogenic molecule. With tumor progression, the number of proangiogenic factors tends to increase (Fidler, 2001, J Natl Cancer Inst 93:1040-1041). This would limit the use of single agents with a narrow spectrum of action and might lead to resistance or tolerance. Indeed, it has been described that tumors eventually become resistant to the antiangiogenic treatment in almost all patients, but the underlying mechanism of resistance has not yet been clarified. Tumors may activate alternative pathways to stimulate the angiogenic process; for example, VEGF inhibition can induce an increase of bFGF pathway in mice. Combination strategies have been therefore evolved to get maximal effect. Experimental models have been used to show that human neuroblastoma cell lines inoculated into immunodeficient mice do not form a tumor if vinblastine and the antiangiogenic agent DC101, a monoclonal antibody targeting VEGFR2 are used in combination (Klement et al., 2000, J Clin Invest 105:R15-R24). Similarly, Kamat and collegues (Cancer Res 2007, 67:281-288) have shown the efficacy of taxanes in combination with AEE788, a dual epidermal growth factor receptor (EGFR) and VEGFR inhibitor, in a murine model of ovarian cancer. Widespread opinion is that angiogenesis has to be simultaneously blocked at different steps of the angiogenic process.

Therefore, there is a critic need for more effective cancer therapies based on the block of angiogenesis by acting at the endothelial cell level, by interfering with intracellular signals that conduit cells to acquire angiogenetic phenotypes and functions.

The availability of new drugs capable of inhibiting angiogenesis opens up interesting applications, possibly for all those diseases where the angiogenic process needs to be regulated.

This is particularly true for drugs showing potent antiangiogenic properties with apparently new mechanisms, by acting from inside the endothelial cell.

As summarized by Rui-Chen in Cancer Metastasis Rev (2006, 25:677-694), in the past few years it has become apparent that lymphangiogenesis, the formation of new lymphatics, ultimately controlled by a complex network of growth factors, cytokines and chemokines can contribute actively to tumour metastasis. Several studies have found positive correlation between lymphangiogenic factors, lymphatic invasion, distant metastasis and in some instances, poor clinical outcomes. The relative role of tumour-derived lymphangiogenic factors, as VEGF-C, is critical for tumour lymphangiogenesis. In fact, recent studies have indicated that the lymphatics undergo dramatic lymphangiogenic changes in response to rapid tumour growth, e.g., lymphatic endothelial cell (LEC) sprouting, intratumoural and peritumoural vessel formation, and dilation of collecting lymphatics adjacent to tumour tissues. The overexpression of VEGF-C by human breast carcinomas has been closely correlated with intratumoural lymphangiogenesis, increased number of intratumoural lymphatic vessels and a high frequency of regional lymph node metastasis. These experiments have provided proof of principle that lymphatics may proliferate within tumours and serve as a conduit for lymphogenic dissemination. Therefore, the targeting of intratumoural and peripheral LECs or blocking lymphangiogenesis have been proposed as an important route for antimetastatic approaches.

There is currently no effective drug able to interfere with lymphangiogenesis and prevent cancer metastasis.

Antilymphangiogenic therapies representing potential treatments for metastatic cancer are an unmet need in the area of oncology.

The availability of new drugs capable of inhibiting lymphangiogenesis opens up interesting applications, possibly for all those diseases where the lymphangiogenic process needs to be regulated.

The optimal drug should exert its antilymphangiogenic activity at the

LEC level, by interfering with intracellular signals that conduit cells to acquire lymphangiogenetic phenotypes and functions.

HHV-6 U94 Gene

HHV-6 U94 gene was described for the first time by Thomson et al (Thomson et al., nature 351:78-80, 1991). The sequence, obtained from the complete HHV-6 DNA sequence (Gompels et al., Virology 209:29-51, 1995), is deposited in RefSeq databank with the geneid 1487994 (National Center for Biotechnology Information, http://www.ncbi.nlm.nih.gov/). U94 is a latency-associated gene, implicated in the regulation of HHV-6 replication (Caseli et al., Virology 346:402-414, 2007).

### Prior Art

Cancer Cell International (2005, 5:19) describes that stable expression of U94 gene in human prostate tumor cell line, named PC3, inhibits its focus formation in culture and tumorigenesis in nude mice. The observed antitumor effect is due to the upregulation of fibronectin (FN1) and reduction of Angiopoietin-like-4 (ANGPTL4) expression in PC3 cells.

Previous studies indicated that U94 suppresses transformation by the oncogene H-ras in NIH3T3 cell line stably expressing U94 (Arauji J C et al., J Virol 1995, 69:4933-4940; Araujio J C et al., Oncogene 1997, 14:937-943).

None of these papers suggests that U94 gene and the encoded Rep protein have normal human primary vascular and lymphatic endothelial cells as a therapeutic target for exerting their anti-(lympho)angiogenic effects.

### DISCLOSURE OF THE INVENTION

The invention is based on the finding that the HHV-6 U94 gene (U94) or the U94 protein product Rep, when delivered to human primary BECs or LECs, impair the physiological capability of cells to form capillary-like structures in vitro.

In a first embodiment, the invention provides the use of Rep protein, analogues thereof or nucleic acid molecules encoding them, for inhibiting endothelial cell function and thereby preventing or treating angiogenesis and/or lymphangiogenesis-related states, conditions or diseases in a mammalian subject in need thereof, preferably in a human subject.

States, conditions or diseases that are mediated by angiogenesis and/or lymphangiogenesis and that may benefit from the treatment according to the invention include, but are not limited to, hemangioma, solid tumours, Kaposis's sarcoma, leukemia, metastasis, telangiectasia, psoriasis, scleroderma, pyogenic granuloma, myocardial angiogenesis, plaque neovascularisation, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, reumathoid arthritis, diabetic neovascularisation, macular degeneration, Wuchereria bancrofti infection, Bartonella infection, wound healing, peptic ulcer, fractures, keloids, vasculogenesis, hematopoiesis, ovulation, menstruation. The treatment consists in the administration of Rep protein, analogues thereof or nucleic acid molecules encoding them to the individual, in amounts sufficient to control the clinical aspects of the condition or disease.

In a further embodiment the invention provides the use of Rep protein, analogues thereof or nucleic acid molecules encoding them for controlling birth, by administering an effective amount of Rep protein, analogues thereof or nucleic acid molecules encoding them to a female such that uterine endometrial vascularisation is inhibited and embryo implantation cannot occur or be sustained.

In a yet further embodiment the invention provides a pharmaceutical preparation for the treatment of the above described diseases or conditions. The pharmaceutical preparation contains Rep protein, analogues thereof or nucleic acid molecules encoding them combined with a pharmaceutically acceptable vehicle. The amount of Rep protein, analogues thereof or nucleic acid molecules encoding them in the dosage form has to be sufficient to substantially lessen manifestation of the disease or to ameliorate symptoms of the state or condition. Manifestation of the disease may be determined by clinical symptoms associated with the disease, and by non invasive and/or invasive instrumental and laboratory methods.

In a yet further embodiment the invention provides the use of Rep protein, analogues thereof or nucleic acid molecules encoding them to inhibit endothelial cell function by promoting or blocking the release of biologically active molecules in the extracellular environment by blood vascular or lymphatic endothelial cells or other cell types.

### DESCRIPTION OF THE DRAWINGS

FIG. 1 shows the presence of U94 transcripts in BECs after nucleofection of pSR2ph-U94 by AMAXA. pSR2ph-U94 plasmid DNA has been used as positive control (C+), whereas amplification of the β-actin cDNA, an house-keeping gene, has been performed to verify that all samples obtained from transfected cells have been correctly processed. Results obtained show that the U94 gene is transcribed in BECs up to 5 days after pSR2ph-U94 transfection.

FIG. 2 shows that pSR2ph-U94 transfected BECs and LECs do not form capillary-like structures at 24 h after seeding on Cultrex® BME as compared to pSR2ph transfected or to not trasfected (NT) cells.

FIG. 3 shows that BECs treated for 24 h with recombinant Rep protein (5 μg/ml) do not form capillary-like structures at 24 h after seeding on Cultrex® BME as compared to untreated cells or to cells treated with heat-inactivated Rep (5 μg/ml).

FIG. 4 shows that pSR2ph-U94 transfected BECs have impaired migratory capability as compared to pSR2ph transfected BECs. In fact, as presented in the lower graph, pSR2ph-U94 transfected BECs reached only 20% sealing ar 8 h after a 200 μl pipette-caused wound scratch, as compared to more than 60% sealing of the pSR2ph transfected BECs.

FIG. 5 Effect of Rep on vasculogenesis. Treatment of aortic rings for 7 days with recombinant Rep, at a concentration of 5 μg/ml, resulted in an almost complete reduction of spontaneous vasculogenesis (panel B) as compared to untreated rings (panel A). Stimulation of aorta rings for 7 days with 20 mg/ml of VEGF strongly increased micro vessel outgrowth (panel C), whereas addition of Rep (5 μg/ml) to rings at the beginning of VEGF stimulation, dramatically decreased the number of micro vessels (panel D).

### DETAILED DESCRIPTION OF THE INVENTION

The present invention provides a therapeutic composition for inhibiting angiogenesis and/or lymphangiogenesis in a mammalian subject in need thereof, preferably in a human subject, said composition containing:

a) a HHV-6 nucleic acid molecule encoding a protein having anti-angiogenic and anti-lymphangiogenic activity, wherein said nucleic acid molecule has a sequence selected from:
i) SEQ ID NO:1 (U94 gene);
ii) a sequence differing from SEQ ID NO:1 due to degeneracy of the genetic code;
iii) a sequence which hybridizes to SEQ ID NO:1 under stringent conditions, particularly at 50° C. or higher and 0.1×SSC (15 mM sodium chloride/1. 5 mM sodium citrate);

and/or

b) an expression vector containing the nucleic acid molecule as above defined;

and/or

c) a Rep protein encoded by the nucleic acid molecule as above defined, preferably the protein of SEQ ID NO:2, or an analogue thereof carrying conservative substitutions;

in amounts capable of inhibiting endothelial cell function.

The HHV6 U94 gene and the Rep protein proved surprisingly able to inhibit in vitro capillary-like structure formation and wound healing repair by human blood vascular endothelial cells and lymphatic endothelial cells.

Antiangiogenic/antilymphangiogenic U94 and/or Rep, analogues, homologues or derivatives thereof, can be combined with a therapeutically effective amount of another molecule which negatively regulates angiogenesis/lymphangiogenesis, such as platelet factor 4, thrombospondin-1, tissue inhibitors of metalloproteases, prolactin, angiostatin, endostatin, bFGF soluble receptor, VEGF soluble receptor, antibodies to VEGF, antibodies to bFGF, transforming growth factor beta, interferon alfa, and placental proliferin-related protein.

Antiangiogenic/antilymphangiogenic molecules of the invention may also be combined with chemotherapeutic agents.

The invention includes analogues of the antiangiogenic/antilymphangiogenic Rep, which can be obtained by altering the protein sequences by substitutions, additions or deletions that provide for functionally equivalent molecules. These include altering sequences in which functionally equivalent amino acid residues are substituted for residues within the sequence resulting in a silent change (conservative substitutions). For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

The antiangiogenic/antilymphangiogenic Rep and analogues thereof can be derived from tissue or produced by various methods known in the art. The operations, which result in their production, can occur at the gene or protein level. For example, a cloned gene sequence coding for antiangiogenic/antilymphangiogenic Rep or analogues thereof can be modified by any of numerous strategies known in the art (Sambrook et al., 1990, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analogue, care should be taken to ensure that the modified gene remains within the same translational reading frame, uninterrupted by translational stop signals.

The antiangiogenic/antilymphangiogenic Rep and analogues thereof are preferably produced by recombinant methods.

The term “isolated” means that Rep is removed from its original environment. For example, a naturally-occurring Rep present in a living individual is not isolated, but the same Rep, separated from some or all of the coexisting materials in the natural system, is isolated. Rep could be part of a vector and/or part of a composition, and still be isolated in that such vector or composition is not part of its natural environment.

Where it is desired to express Rep, any suitable system can be used. The general nature of suitable vectors, expression vectors and constructions therefor will be apparent to those skilled in the art.

Suitable expression vectors may be based on phages or plasmids, both of which are generally host-specific, although these can often be engineered for other hosts. Other suitable vectors include cosmids and retroviruses, and any other vehicles, which may or may not be specific for a given system. Control sequences, such as recognition, promoter, operator, inducer, terminator and other sequences essential and/or useful in the regulation of expression, will be readily apparent to those skilled in the art.

The DNA encoding Rep or a Rep-like protein may readily be inserted into a suitable vector. Ideally, the receiving vector has suitable restriction sites for ease of insertion, but blunt-end ligation, for example, may also be used, although this may lead to uncertainty over reading frame and direction of insertion. Suitable vectors may be selected as a matter of course by those skilled in the art according to the expression system desired.

By transforming a suitable organism or eukaryotic cell line with the plasmid obtained, selecting the transformant with ampicillin, kanamicin or by other suitable means if required, and adding tryptophan or other suitable promoter-inducer (such as indoleacrylic acid) if necessary, Rep may be expressed. The extent of expression may be analyzed by SDS polyacrylamide gel electrophoresis-SDS-PAGE and by Western blot analysis.

Suitable methods for growing and transforming cultures etc. are usefully illustrated in, for example, Molecular Cloning, A Laboratory Manual (2nd Ed., Sambrook, Fritsch and Maniatis, Cold Spring Harbor), Current Protocols in Molecular Biology (Eds. Aufubel, Brent, Kingston, More, Feidman, Smith and Stuhl, Greene Publ. Assoc., Wiley-Interscience, NY, N.Y. 1992). Rep may be isolated from the fermentation or cell culture and purified using any of a variety of conventional methods including: liquid chromatography such as normal or reversed phase, using HPLC and FPLC; affinity chromatography (such as with inorganic ligands or antibodies); size exclusion chromatography; immobilized metal chelate chromatography; gel electrophoresis. One of skill in the art may select the most appropriate isolation and purification techniques without departing from the scope of this invention.

Rep may be generated by any of several chemical techniques. For example, it may be prepared using the solid-phase synthetic technique originally described by R. B. Merrifield, “Solid Phase Peptide Synthesis. I. The Synthesis Of A Tetrapeptide”, J. Am. Chem. Soc., 83, pp. 2149-54 (1963), or they may be prepared by synthesis in solution. A summary of peptide synthesis techniques may be found in E. Gross & H. J. Meinhofer, The Peptides: Analysis, Synthesis, Biology; Modern Techniques Of Peptide And Amino Acid Analysis, John Wiley & Sons, (1981) and M. Bodanszky, Principles Of Peptide Synthesis, Springer-Verlag (1984).

The functional activity and/or therapeutically effective dose of Rep or nucleic acid encoding therefor can be assayed in vitro by various methods. For example, where one is assaying for the ability of the angiogenic inhibitory Rep to inhibit or interfere with the capability of endothelial cells to form capillary-like structure on extracellular matrices in vitro, various bioassays known in the art can be used, including, but not limited to, wound healing, inhibition of endothelial cell proliferation, inhibition of endothelial cell migration and cell counting.

Assays for the ability to inhibit angiogenesis in vivo include the chick chorioallantoic membrane assay and mouse, rat or rabbit corneal pocket assays.

The ability of the antiangiogenic protein to influence angiogenesis can also be determined using a number of known in vivo and in vitro assays. Such assays are disclosed in Jain et al., (Nature Med 1997, 3:1203-1208).

The therapeutically effective dosage for inhibition of angiogenesis in vivo, defined as inhibition of capillary-like formation of extracellular matrices and of endothelial cell proliferation and migration, may be extrapolated from in vitro inhibition assays using the compositions of the invention above or in combination with other angiogenesis inhibiting factors. The effective dosage is also dependent on the method and means of delivery. For example, in some applications, as in the treatment of psoriasis or diabetic retinopathy, the inhibitor is delivered in a topical-ophthalmic carrier. In other applications, as in the treatment of solid tumors, the inhibitor is delivered by means of a biodegradable, polymeric implant. The protein can also be modified, for example, by polyethyleneglycol treatment.

Diseases, disorders, or conditions, associated with abnormal angiogenesis or neovascularization, that can be treated with a therapeutic composition of the invention include, but are not limited to retinal neovascularization, tumor growth, hemagioma, solid tumors, leukemia, metastasis, psoriasis, neovascular glaucoma, diabetic retinopathy, arthritis, rheumatoid arthritis. endometriosis, and retinopathy of prematurity ROP).

As used herein. the term “effective dosage” refers to an amount of the antiangiogenic protein of the invention sufficient to exhibit a detectable therapeutic effect. The therapeutic effect may include, for example, without limitation, inhibiting the growth of undesired tissue or malignant cells, inhibiting inappropriate angiogenesis (neovascularization), limiting tissue damage caused by chronic inflammation, inhibition of tumor cell growth. The precise effective amount for a subject will depend upon the subject's size and health, the nature and severity of the condition to be treated. Thus, it is not possible to specify an exact effective amount in advance. However, the effective amount for a given situation can be determined by routine experimentation based on the information provided herein.

The antiangiogenic protein of the invention is administered orally, topically, or by parenteral means, including subcutaneous and intramuscular injection, implantation of sustained release depots, intravenous injection or intranasal administration. Accordingly, the antiangiogenic protein of the invention is preferably administered as a pharmaceutical composition comprising the antiangiogenic protein of the invention in combination with a pharmaceutically acceptable carrier. The term “pharmaceutically acceptable” refers to compounds and compositions which may be administered to mammals without undue toxicity. Exemplary pharmaceutically acceptable salts include mineral acid salts such as hydrochlorides, hydrobromides, phosphates and sulfates; and the salts of organic acids such as acetates, propionates, malonates or benzoates. Such compositions may be aqueous solutions, emulsions, creams, ointments, suspensions, gels and liposomal suspensions. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils. One may additionally include suitable preservatives, stabilizers, antioxidants, antimicrobials, and buffering agents, for example, BHA, BHT, citric acid, ascorbic acid and tetracycline. Cream or ointment bases useful in formulation include lanolin, Silvadene® (Marion), Aquaphor® (Duke Laboratories). Other topical formulations include aerosols, bandages, and other wound dressings. Alternatively one may incorporate or encapsulate the therapeutic compound of the invention in a suitable polymer matrix, nanobubble or membrane, thus providing a sustained-release delivery device suitable for implantation near the site to be treated locally. Other devices include indwelling catheters and devices such as the Alzet® minipump. Ophthalmic preparations may be formulated using commercially available vehicles such as Sorbi-care® (Allergan), Neodecadron® (Merck, Sharp & Dohme), Lacrilube®, or may employ topical preparations such as that described in U.S. Pat. No. 5,124,155, incorporated herein by reference. Further, one may provide a therapeutic compound of the invention in solid form, especially as a lyophilized powder. Lyophilized formulations typically contain stabilizing and bulking agents, for example human serum albumin, sucrose and mannitol. A thorough discussion of pharmaceutically acceptable excipients is available in Remington's Pharmaceutical Sciences (Mack Pub. Co.).

The DNA encoding the antiangiogenic protein of the invention can be used in the form of gene therapy and delivered to a host by any method known to those of skill in the art to treat disorders associated with angiogenic or tissue remodeling-associated conditions.

A preferred embodiment of the present invention relates to methods of inhibiting angiogenesis of solid tumors to prevent further tumor growth and metastasis. To this end, any solid tumor or the region surrounding the tumor accessible to gene transfer will be a target for the disclosed therapeutic applications. A DNA encoding an angiogenic polypeptide, housed within a recombinant viral- or non-viral-based gene transfer system may be directed to target cells within proximity of the tumor by a number of procedures known in the art, including but not limited to (a) surgical procedures coupled with administration of an effective amount of the DNA to the site in and around the tumor (involving initial removal of a portion or the entire tumor, if possible); (b) injection of the gene transfer vehicle directly into or adjacent to the site of the tumor; and, (c) localized or systemic delivery of the gene transfer vector and/or gene product using techniques known in the art. Any solid tumor will be a potential target for treatment. Examples, but by no means intended as a limitation, of solid tumors which will be particularly vulnerable to gene therapy applications are (a) neoplasms of the central nervous system such as, but again not limited to glioblastomas, astrocytomas, neuroblastomas, meningiomas, ependymomas; (b) cancers of hormone-dependent, tissues such as prostate, testicles, uterus, cervix, ovary, mammary carcinomas including but not limited to carcinoma in situ, medullary carcinoma, tubular carcinoma, invasive (infiltrating) carcinomas and mucinous carcinomas; (c) melanomas, including but not limited to cutaneous and ocular melanomas; (d) cancers of the lung which at least include squamous cell carcinoma, spindle carcinoma, small cell carcinoma, adenocarcinoma and large cell carcinoma; and (e) cancers of the gastrointestinal system such as esophageal, stomach, small intestine, colon, colorectal, rectal and anal region which at least include adenocarcinomas of the large bowel.

In a particularly preferred embodiment of the invention, the U94 gene, the encoded Rep protein or analogues thereof according to the invention are used in the preventive and therapeutic treatment of tumors that display the capability to differentiate in endothelial-like cells and form per se capillary-like structures on suitable matrices. These include human breast adenocarcinoma (Serwe et al., Invest New Drugs, Nov. 14, 2008, E-pub ahead of print) and human melanoma (Mourad-Zeidan et al., Am. J. Pathol. 173: 1839-52, 2008), which promote transformation of their cancer elements into endothelial-like cells able to perform angiogenesis and support tumor survival and progression as well as methastatic diffusion. This is a distinctive feature of the invention, as the therapeutic applications so far envisaged for U94 do not allow to predict its specific effectiveness in the treatment of tumors characterized by elements with angiogenetic capability.

The DNA encoding the antiangiogenic protein may be delivered either systemically or to target cells in the proximity of a solid tumor of the mammalian host by viral or non-viral based methods. Viral vector systems which may be utilized in the present invention include, but are not limited to, adenovirus vectors; retrovirus vectors; adeno-associated virus vectors; herpes simplex virus vectors; SV 40 vectors; polyoma virus vectors; papilloma virus vectors; picornavirus vectors; and vaccinia virus vectors.

The recombinant virus or vector containing the DNA encoding the antiangiogenic protein of the present invention is preferably administered to the host by direct injection into a solid tumor and/or quiescent tissue proximal to the solid tumor, such as adipose or muscle tissue. It will of course be useful to transfect tumor cells in the region of targeted adipose and muscle tissue. Transient expression of the antiangiogenic polypeptide in these surrounding cells will result in a local extracellular increase in these proteins.

Non-viral vectors which may be utilized in the present invention include, but are not limited to, DNA-lipid complexes, for example liposome-mediated or ligand/poly-L-Lysine conjugates, such as asialoglyco-protein-mediated delivery systems (see, e.g., Feigner et al., 1994, J. Biol. Chem. 269:2550-2561; Derossi et al., 1995, Restor. Neurol. Neuros. 8:7-10; and Abcallah et al., 1995, Biol. Cell 85:1-7; Karmali and Chaudhuri, 2006, Med Res Rev 27:696-722), biodegradable polymers (see, e.g., Luten et al., 2008, J Control Release 126:97-110) or microbubbles loaded with plasmid DNA (see, e.g., Bekeredjian et al., 2003, Circulation 108:1022-1026). Direct injection of “naked” DNA may also be used (see, e.g., Lin et al., Circulation 1990, 82:2217-2221).

The invention also provides a pharmaceutical pack or kit comprising one or more containers filled with one or more of the ingredients of the pharmaceutical compositions of the invention. Optionally associated with such container(s) can be a notice or leaflet in the form prescribed by a governmental agency regulating the manufacture, use or sale of pharmaceuticals or biological products, which notice reflects approval by the agency of manufacture, use or sale for human administration. The references cited throughout this application are herein incorporated by reference. The present invention is further illustrated by the following Examples.

### EXAMPLES

The following examples further illustrate the invention. These examples are not intended to limit the scope of the invention. In light of the present disclosure, numerous embodiments within the scope of the claims will be apparent to those of ordinary skill in the art.

### Example 1

Primary Human Endothelial Cell Transfection

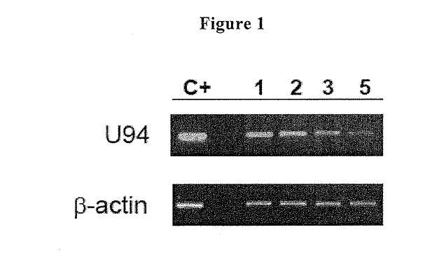

The full-length U94 gene was obtained by PCR amplification of ORF U94 of HHV-6 variant B, as previously described (Rotola et al., 1998, Proc Nati Acad Sci USA 95:13911-13916). The U94 gene from HHV-6B R1 strain (Dewhurst et al., 2000, Virology 190:490-493) was cloned in the pSR2ph vector (pSR2ph-U94). The pSR2ph-U94 (2 was nucleofected into 10⁶ BECs or LECs using the AMAXA device (Koln, Germany) following the manufacturer's instructions. Cells were harvested at different time points after the transfection (1,2,3,and 5 days) and total RNAs were extracted. Following retrotranscription, the cDNA was amplified by PCR using U94 specific primers as described (Rotola et al., 1998, Proc Nati Acad Sci USA 95: 13911-13916). As shown in FIG. 1, U94 gene is constantly expressed in BECs as demonstrated by the presence of U94 transcripts at each time point evaluated. Similar results were obtained using LECs upon pSR2ph-U94 transfection. As positive control of the reaction we used pSR2ph-U94 plasmid, whereas amplification of the housekeeping gene β-actin attested for the suitability of cDNA samples.

### Example 2

Cord Formation of pSR2ph-U94-Ttransfected Endothelial Cells on Culture Basement Membrane Extract (BME)

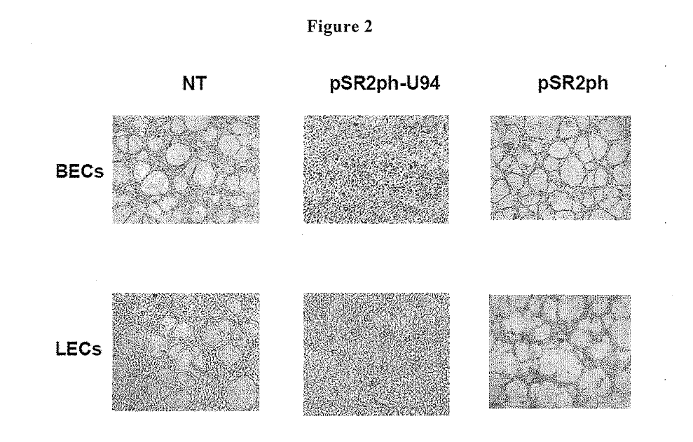

Two hundred microliters of BME (10 mg/ml) (Cultrex®; Trevigen Inc., Gaithersburg, Mass.) at 4° C. were transferred to pre-chilled 48-well culture plates using sterile tips that had been cooled at −20° C. before use. After gentle agitation to ensure coating, the plates were incubated for lh at 37° C. to allow Cultrex® BME to solidify. Primary BECs or LECs were then seeded at a concentration of 6×10⁴/well in EGM. Cord formation was observed at 24 h after cell seeding. Both endothelial cell types spontaneously form, in the presence of Endothelial Growth Medium (EGM) containing VEGF, capillary-like structures on Cultrex® BME at 24 h after seeding. As representatively shown in FIG. 2, BECs and LECs harvested at day 1 after pSR2ph-U94 transfection and cultured on Cultrex® in the presence of EGM, were unable to form tubes at 24 h after seeding. In particular, we observed that cells adhered to Cultrex® BME forming a monolayer with the formation of few hollow tube-like structures. On the contrary, BECs or LECs transfected with the pSR2ph empty plasmid, maintained the capability to form a complex network of capillary-like structures.

### Example 3

Cord Formation of Rep-Treated Endothelial Cells on Culture Basement Membrane Extract (BME)

The Rep coding gene was excised from pSR2ph by HindIII digestion and subcloned into bacterial pQE30 vector (Qiagen), obtaining the recombinant plasmid pQE-rep. The gene was cloned in frame with a stretch of six histidine residues (His)6 at the amino-terminus, under the control of a T5 promoter/lac operator. The recombinant plasmid was used to transform the M15 E. coli strain, harboring the pREP4 plasmid, which contains the lad gene coding for lac repressor. Upon addition of isopropylthiogalactoside (IPTG), the T5/lac promoter is activated, resulting in high yield production of the fusion protein cloned in the pQE30. Rep protein was produced and purified, in the absence of endotoxin contamination, as already described (Caselli et al., 2006, Virology 346:402-414).

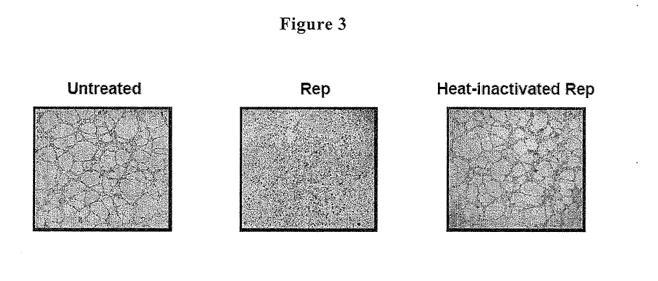

Primary BECs or LECs were treated with recombinant Rep at doses ranging from 0.1 to 5 μg/ml for 24 h and resuspended in EGM. Cells were then seeded at a concentration of 6×10⁴ cells/well on BME-coated 48-well plates. Untreated cells showed cord formation at 24 h after cell seeding. BECs or LECs harvested at day 1 after Rep treatment and cultured on Cultrex® in the presence of EGM, were unable to form tubes at 24 h after seeding. The recombinant Rep inhibited BECs or LECs angiogenic activity in a dose-dependent manner, with optimal inhibition observed at a protein concentration of 5 μg/ml. To confirm specificity, experiments were also performed using equal amounts (5 μg/ml) of heat denaturated Rep obtained by boiling the protein preparation at 100° C. for 10 minutes (FIG. 3). The results show that heat denaturation abolished Rep activity, suggesting that folding of the protein is important for its action.

### Example 4

Cord Formation of pSR2ph-U94-Transfected MDA-MB-231 Cells on Culture Basement Membrane Extract (BME)

The pSR2ph-U94 (2 μg) was nucleofected into 10⁶ MDA-MB-231 cells (ATCC Number HTB-26: derived from a human breast adenocarcinoma) using the AMAXA device following the manufacturer's instructions. MDA-MB-231 cells were then seeded at a concentration of 6×10⁴/well in RPMI-1640 medium. Cells spontaneously formed, in the absence of any pro-angiogenetic factor in the medium, capillary-like structures on Cultrex® at 24 h after seeding. Cells harvested at day 1 after pSR2ph-U94 transfection and cultured on Cultrex® were unable to form tubes at 24 h after seeding. On the other hand, cells transfected with the pSR2ph empty plasmid maintained the capability to form a complex network of capillary-like structures.

### Example 5

Endothelial Cell Migration Assay

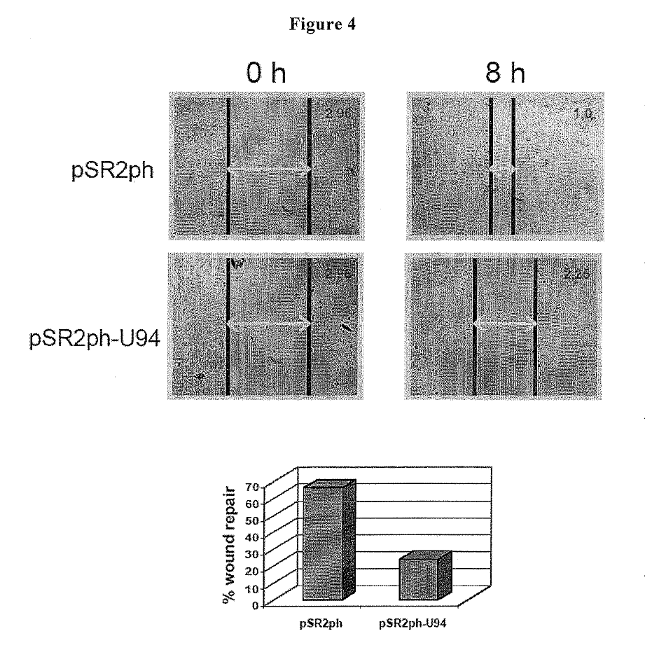

The pSR2ph-U94 (2 μg) was nucleofected into 10⁶ BECs or LECs using the AMAXA device (Koln, Germany) following the manufacturer's instructions. Cells were harvested at 24 h after the transfection, seeded (2.5×10⁵ cells/well) on collagen-coated 12-well culture plates and grown to confluence. At the same time, primary BECs or LECs were treated for 24 h with recombinant Rep (5 μg/ml), seeded as above on collagen-coated 12-well culture plates and grown to confluence. Confluent monolayers were scratched using either a 200 μl or 1 ml pipette tip. Endothelial cell migration was observed by optical microscopy (Leica DM IRB, Wetzlar, Germany) and photographed with CCD optics (Hitachi Denshi Color Camera KP-D50E/K; Rodgau, Germany) using a digital analysis system (QWIN LITE version 2.3; Leica). The percentage of wound sealing was observed over an 8 -h time course. As shown in FIG. 4, control BECs reached 60-80% sealing at 8 h after the 200 μl pipette-caused wound scratch. The pSR2ph-U94 transfected BECs, scratched at the same time of control BECs, only showed about 20-30% sealing 8 h after wound scratch. Similar results were obtained using pSR2ph-U94 transfected LECs. These results suggest that U94 gene expression in cells attenuated the migration of BECs and LECs in the wound healing assay.

The same assay was run using BECs and LECs treated or not for 24 h with recombinant Rep protein, at a concentration of 5 μg/ml. Cell migration was strongly affected in Rep-treated BECs or LECs as compared to their untreated counterpart. In fact, Rep-treated cells showed only 10% of the wound sealing activity of control cells 8 h after wound scratch.

### Example 6

Aortic Ring Assay

The rat aorta ring assay was performed as previously described (Nicosia R. F. and Ottinetti A. 2000. Lab. Invest. 63: 115-122). Briefly, rats were sacrificed by CO2 saturated atmosphere, the dorsal aorta was excised and cleaned from fat under dissection microscopy. Rings, approximately lmm thick, were prepared and stored in DMEM at 4° C. for less than 2 hours before use. Eight volumes of type 1 collagen solution (1.5 mg/ml) prepared from rat tail were mixed with 1 volume of 10× DMEM and 1 volume of NaHCO3 (23.4 mg/ml). Forty μl of collagen solution were placed in each well of 4-well plates and one aortic ring was transferred in each well. The plates were incubated in humidified atmosphere at 37° C. for 30 minutes to obtain jellification. After incubation each well was filled with 500 μl of EBM containing recombinant Rep (0.5-5 μg) alone or in combination with 20 ng/ml of VEGF. Plates were incubated for 10-12 days. Quantification of angiogenesis was obtained by taking photographs at three days intervals and counting the number of vessels originating from the aortic rings.

### Example 7

Rep Blocks Vasculogenesis of Rat Aortic Rings

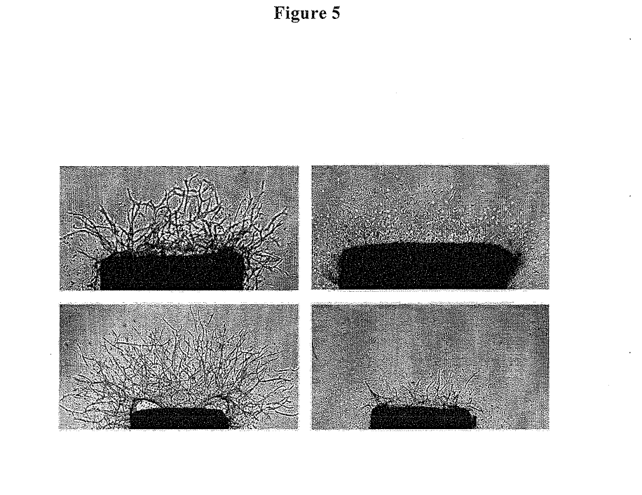

The effect of recombinant Rep on vasculogenesis was studied also using the aorta ring assay. This method permits to investigate the ability of molecules to interfere with the in vitro growth of mammal micro vessels. Treatment of aortic rings with recombinant Rep resulted in a sharp reduction of spontaneous micro vessels outgrowth. Maximal inhibition of vasculogenesis induced by Rep was observed after 7 days of incubation at a protein concentration of 5 μg/ml. At this time, the number of micro vessels in control cultures were 45 ±18 compared to 5±4 in rings treated with Rep (FIG. 5). As expected, stimulation of aorta rings with 20 mg/ml of Vascular Endothelium Growth Factor (VEGF) strongly increased micro vessel outgrowth (285±74). Addition of Rep (5 μg/ml) to rings treated with VEGF decreased the number of micro vessels to 83±29 (FIG. 5). Lower concentrations of U94 (2.5-1 μg/ml) were still inhibitory, but less effective. Rep, at the concentration of 0.5 μg/ml did not affect rat aorta angiogenesis. This result shows that Rep is not only capable of interfering with mechanisms underlying spontaneous vasculogenesis, but also renders aortic rings completely insensitive to the potent VEGF-induced vasculogenetic activity.

1-12. (canceled)

13. A method for preventing or treating an angiogenesis and/or lymphangiogenesis-related disease, state or condition in a mammalian subject, comprising administering to the mammalian subject a therapeutically effective amount of a composition containing, alone or in combination:
a) a HHV-6 nucleic acid molecule encoding a protein having anti-angiogenic and anti-lymphangiogenic activity, wherein said nucleic acid molecule has a sequence selected from:
i) SEQ ID NO:1;
ii) a sequence differing from SEQ ID NO:1 due to degeneracy of the genetic code;
iii) a sequence which hybridizes to SEQ ID NO:1 under stringent conditions;
b) an expression vector containing the nucleic acid molecule as above defined;
c) a protein encoded by the nucleic acid molecule as above defined or an analogue thereof carrying conservative substitutions and retaining anti-angiogenic and anti-lymphangiogenic activity.

14. The method according to claim 13, wherein said mammalian subject is human.

15. The method according to claim 13, wherein in said composition, said nucleic acid molecule is in the form of the naked DNA.

16. The method according to claim 13, wherein in said composition, said expression vector is a viral vector.

17. The method according to claim 16, wherein said viral vector is—selected from one of the following: adenovirus vector, retrovirus vector, adeno-associated virus vector, herpes simplex virus vector, SV 40 vector, polyoma virus vector, papilloma virus vector, picornavirus vector and vaccinia virus vector.

18. The method according to claim 13, wherein in said composition, said protein encoded by the nucleic acid molecule has the sequence of SEQ ID NO: 2.

19. The method according to claim 18, wherein said protein is in the form of a purified recombinant protein.

20. The method according to claim 13, wherein said composition is in the form of DNA-lipid complex, liposome-mediated or ligand/poly-L-Lysine conjugate, asialoglyco-protein-mediated delivery system, biodegradable polymer or microbubble loaded with plasmid DNA.

21. The method according to claim 13, wherein the angiogenesis and/or lymphangiogenesis-related disease, state or condition is selected from the group consisting of: hemangioma, solid tumours, Kaposis's sarcoma, leukemia, metastasis, telangiectasia, psoriasis, scleroderma, pyogenic granuloma, myocardial angiogenesis, plaque neovascularisation, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, reumathoid arthritis, diabetic neovascularisation, macular degeneration, Wuchereria bancrofti infection, Bartonella infection, wound healing, peptic ulcer, fractures, keloids, vasculogenesis, hematopoiesis, ovulation and menstruation.

22. The method according to claim 13, wherein the angiogenesis and/or lymphangiogenesis-related disease, state or condition is a tumor that displays the capability to differentiate in endothelial-like cells and form capillary-like structures.

23. The method according to claim 13, wherein the angiogenesis and/or lymphangiogenesis-related disease, state or condition is selected from human breast adenocarcinoma and human melanoma.

24. The method according to claim 13, wherein the angiogenesis and/or lymphangiogenesis-related disease, state or condition is uterine endometrial vascularisation or embryo implantation.


SEQUENCE LISTING (2 sequences)

SEQ ID NO: 1 - DNA, 1473 bp, Human herpesvirus 6
atgttttccataataaatccgagtgatgatttctggactaaggacaaatatatcatgttgactatcaaaggccccatggagtgggaggcagaaatccctggaatatctacggattttttctgtaaattctctaacgtgtccgtgccacatttcagggatatgcactcaccgggagcgcccgatattaaatggataactgcatgtaccaaaatgatcgatgtcatactcaattactggaataataaaactgccgtccccacccctgcaaagtggtacgctcaagcggagaataaagctggcagaccctccctaatattattgatagctttagatggaattcccagcgcaacgataggaaaacacacaacggaaatcaggggcgtattaattaaagatttcttcgacgggaacgcccctaaaatagatgattggtgcacgtatgccaaaacaaagaaaaatggtggcggaacccaggtcttcagtctaagttatatcccctttgcccttcttcaaattattagaccacagttccaatgggcgtggacaaatattaacgaactgggagacgtatgcgatgaactacatcgaaaacacatcatatcccattttaataaaaaacctaatgtcaaacttatgctgtttccaaaggatgggattaacgggatatctttaaaatctaaatttctgggaaccatcgaatggctgtctgatcttggaatagtcacggaagacgcgtggatacgaagagacgttagatcatacatgcaattattgacgctaacacacggggacgtgctaatccatagggctctatctatatctaaaaaaagaataagagcaactagaaaagctatcgattttatagcgcacatagacactgactttgaaatctatgaaaacccggtttaccagttgttctgtctgcagtcttttgaccctatattagcaggaaccatattatatcagtggctaagccacagaagagggaaaaaaaacaccgttagttttatcggtccacccggatgtggaaaatcgatgttaacgggagccattcttgaaaatatcccgttacatggaatattacacggatctttgaatactaaaaatttaagagcttacggacaggttttagtcttgtggtggaaagacataagtatcaactttgaaaattttaatattataaaatccctccttgggggtcaaaaaataatattcccaattaatgaaaacgaccacgtacagataggaccgtgtcccatcatagccacatcttgcgttgatatacgctcgatggtacattccaatatccacaaaataaatctatcacagagggtatataatattacatttgaaaaagttatccctcgcaattttcctgtaattcagaaagacgacataaatcaatttctgttctgggccagaaaccgttctataaattgttttattgactacacggttccgaaaattttataa

SEQ ID NO: 2 - protein, 490 aa, Human herpesvirus 6
MFSIINPSDDFWTKDKYIMLTIKGPMEWEAEIPGISTDFFCKFSNVSVPHFRDMHSPGAPDIKWITACTKMIDVILNYWNNKTAVPTPAKWYAQAENKAGRPSLILLIALDGIPSATIGKHTTEIRGVLIKDFFDGNAPKIDDWCTYAKTKKNGGGTQVFSLSYIPFALLQIIRPQFQWAWTNINELGDVCDELHRKHIISHFNKKPNVKLMLFPKDGINGISLKSKFLGTIEWLSDLGIVTEDAWIRRDVRSYMQLLTLTHGDVLIHRALSISKKRIRATRKAIDFIAHIDTDFEIYENPVYQLFCLQSFDPILAGTILYQWLSHRRGKKNTVSFIGPPGCGKSMLTGAILENIPLHGILHGSLNTKNLRAYGQVLVLWWKDISINFENFNIIKSLLGGQKIIFPINENDHVQIGPCPIIATSCVDIRSMVHSNIHKINLSQRVYNITFEKVIPRNFPVIQKDDINQFLFWARNRSINCFIDYTVPKIL